(19) United States
(12) Patent Application Publication
(10) Pub. No.: US 2019/0008757 A1
(43) Pub. Date: Jan. 10, 2019

(54) DERMATOLOGICAL OR COSMETIC COMPOSITIONS CONTAINING A POLYPHENOL?ENRICHED EXTRACT OF VITEX NEGUNDO

(71) Applicant: Laboratoires Expanscience, Paris La Défense Cedex (FR)

(72) Inventors: Sophie LECLERE-BIENFAIT, Dreux (FR); Philippe MSIKA, Versailles (FR)

(73) Assignee: Laboratoires Expanscience, Paris La Défense Cedex (FR)

(21) Appl. No.: 16/073,728

(22) PCT Filed: Jan. 27, 2017
(86) PCT No.: PCT/EP2017/051826

§ 371 Date: Jul. 27, 2018

(30) Foreign Application Priority Data
Jan. 29, 2016 (FR) 1650752
Apr. 1, 2016 (FR) 1652896

(51) Int. Cl. A61K 8/9789; A61K 8/368; A61K 8/37; A61Q 19/00; A61Q 19/08

(52) U.S. Cl. CPC A61K 8/9789; A61K 8/368; A61Q 19/08; A61Q 19/00; A61K 8/375

(57) ABSTRACT

The invention relates to a cosmetic or dermatological composition containing a polyphenol-enriched extract of Vitex Negundo, typically a root extract obtained using a PAT plant milking®process. The invention further relates to a composition of this kind for use in the prevention or treatment of disorders and diseases affecting the skin, mucous membranes or skin appendages, for use in the prevention or treatment of vascular disorders and for use in the prevention or treatment of extrinsic or intrinsic skin aging. Lastly, the invention relates to a cosmetic care method for the skin, skin appendages or mucous membranes, with a view to improving the condition or appearance thereof, said method consisting in administering such a composition or such an extract.

The present invention relates to a cosmetic or dermatological composition containing a polyphenol-enriched extract of Vitex negundo. The composition of the invention is useful advantageously in the prevention or treatment of disorders or diseases affecting the skin, mucous membranes or skin appendages, and in the prevention or treatment of vascular disorders.

Vitex negundo is commonly known as the Chinese chaste tree, and is called Muguet bleu in France, nôti in Réunion, Chinduravam Notchi in Tamil, or Huang Ping in Chinese, for example. Vitex negundo is a shrub native to India belonging to the family Verbenaceae or Lamiacea, reaching one to two metres in height, with small blue to violet flowers three to five millimetres in size. The leaves, the part most commonly used in traditional medicine, consist of three or five leaflets of lanceolate shape. This plant, which is widely distributed throughout the world, is found in Afghanistan, Pakistan, Sri Lanka, East Africa and Madagascar, and is widely cultivated in Asia, Europe and North America.

The phytochemistry of Vitex negundo has been described by Vishal R. Tandon (Natural Product Radiance 2005, 4(3), 162-165) and Vishwanathan et al. (EJBS 2010, 3(1), 30-42), and is summarised in the table below.

| Plant part | Phytochemical constituents |
|---|---|
| Leaves | Hydroxy-3,6,7,3′,4′-pentamethoxyflavone, 6′-p- |
| | hydroxybenzoyl mussaenosidic acid; 2′-p-hydroxybenzoyl |
| | mussaenosidic acid; Flavonoids (luteolin, vitexin); |
| | vividiflorol, b-caryophyllene, caryophyllene oxide, |
| | sabinene, 4-terpineol, betulinic acid, ursolic acid, |
| | protocatechuic acid, vitamin C, nishindine, p- |
| | hydroxybenzoic acid, beta-sitosterol |
| Seeds | Vitedoine-A, vitedoine B, hydroxybenzoic acid, |
| | beta-sitosterol, n-nonacosane |
| Roots | Vitexin, iso-vitexin, negundins A and B, sitosterol |
| Essential | Delta-guaiene, guaiai-3, 7-dienecaryophyllene epoxide, |
| oil of fresh | ethyl-hexadecenoate, alpha-selinene, germacren-4-ol, |
| leaves, | caryophyllene epoxide, nerilidol, beta-selinene, alpha- |
| flowers and | cedrene, germacrene D, hexadecanoic acid, p-cymene, |
| dried fruits | valencene |

Extracts of various parts of the Vitex negundo plant grown or obtained in its natural environment have numerous pharmacological properties. In particular, the following have been identified:
antalgic and analgesic properties, notably of the leaf and root extract administered in the peritoneum;
antiinflammatory properties, particularly against arthritis and arthrosis, of the leaves, roots and seeds;
antifungal effects of an ethanolic leaf extract consisting of flavonoids (Sathiamoorthy et al., Bioorg Med. Chem. Lett. 2007, 17(1), 239-242);
antibacterial effects on certain bacterial strains, of the essential oil and of ethanolic or ethyl acetate extracts.

In addition, Vitex negundo is used in Reunion in the treatment of chikungunya outbreaks (S. SAVRIAMA—Ethnopharmacologia, no. 52, 2014).

Traditionally, Vitex negundo is often used in decoction, mainly of leaves, and more rarely of roots.

In Ayurvedic rites, the Vitex negundo plant is used for its deworming and anthelminthic properties.

However, all these uses involve the destruction of all or part of the plant. But new regulations aim to control access to biodiversity and sharing of the benefits derived therefrom. In particular, the Nagoya Protocol and the international standard (ISO 26000) provide guidelines considered the benchmark in terms of sustainable development and social responsibility.

Furthermore, the “traditional” Vitex negundo extracts have a phytochemical composition, i.e., a concentration of active principles, and notably of polyphenols, which is not optimal for cosmetic or dermatological applications. In particular, Vitex negundo extracts with a high content of flavonoids have been described in CN 104586700 (flavones content of 82.5% or higher), and by Huang et al. (Journal of Pharmaceutical and Biomedical Analysis, 2015, 108, 11-20).

There is therefore a need for new cosmetic and/or dermatological compositions containing a Vitex negundo extract optimised in cosmetic and/or dermatological terms, which also respects the principles of sustainable development in social and environmental terms.

The Applicant discovered that polyphenol-enriched extracts of Vitex negundo, obtained notably by aeroponic cultivation according to a variant of the Plant Milking® technology of the company Plant Advanced Technologies (technology described in particular in international application WO 01/33942), have a phytochemical composition and cosmetic and dermatological properties which have never before been described. In particular, it is the first time that such Vitex negundo extracts are used as such, for their specific properties.

Indeed, they contain at least 5% by weight of polyphenols, expressed as gallic acid equivalent, relative to the total weight of the dry extract, which is higher than Vitex negundo extracts obtained from plants grown in soil. Nonetheless, the extracts of the invention contain little or no flavonoids.

Furthermore, it has been demonstrated that Vitex negundo, cultivated under these particular aeroponic conditions, with notably an appropriate nutrient solution and an appropriate elicitor, does not produce the molecules usually described in the prior art for this plant, notably the widely described flavonoids vitexin and derivatives thereof, unlike mother plants grown under usual soil conditions.

The Applicant further discovered that these polyphenol-enriched extracts of Vitex negundo have anti-inflammatory, antioxidant and anti-pollution properties. However, inflammatory phenomena, radical attacks, phenomena amplified by pollution and UV exposures are at the origin of chronological and actinic ageing phenomena. The polyphenol-enriched extracts of Vitex negundo and the cosmetic and dermatological compositions containing the same are therefore useful, for example, as antiageing treatment, notably against chronological and actinic ageing of the skin, mucous membranes or skin appendages.

The present invention thus relates to a cosmetic or dermatological composition containing a polyphenol-enriched extract of Vitex negundo as active principle, and, if need be, an appropriate excipient.

The present invention also relates to a cosmetic or dermatological composition containing a polyphenol-enriched extract of Vitex negundo, for use in the prevention or treatment of disorders or diseases of the skin, mucous membranes or skin appendages, and in the prevention or treatment of vascular disorders.

The present invention also relates to a cosmetic care method for the skin, skin appendages or mucous membranes, for improving the condition or appearance thereof, comprising or consisting in the administration of a composition of the invention.

Definitions

The expression “polyphenol-rich extract of Vitex negundo” or “polyphenol-enriched extract of Vitex negundo”, within the meaning of the present invention, refers to a Vitex negundo extract containing at least 5% by weight, for example between 5% and 10%, of polyphenols, expressed as gallic acid equivalent, relative to the total weight of the dry extract. These percentages are notably obtained by a Folin-Ciocalteu assay.

In a Folin-Ciocalteu assay, all phenolic compounds are oxidised by the Folin-Ciocalteu reagent (available commercially). The latter comprises a mixture of phosphotungstic acid (H₃PW₁₂O₄₀) and phosphomolybdic acid (H₃PMo₁₂O₄₀) which is reduced, upon oxidation of the phenolic substances, to a mixture of blue oxides of tungsten (W₃O₂₃) and molybdenum (Mo₈O₂₃). The blue colouring produced has a maximum absorption around 750-760 nm. It is proportional to the amount of oxidised phenolic compounds. The reference phenol used in this method is gallic acid. The results obtained by this assay are therefore expressed as “% by weight of polyphenols, expressed as gallic acid equivalent, relative to the total weight of the dry extract”. Nonetheless, the “polyphenol-rich extract of Vitex negundo” or the “polyphenol-enriched extract of Vitex negundo” contains little or no flavonoids. Flavonoids are a subclass of polyphenols, having an optionally substituted 15-carbon skeleton, said 15-carbon skeleton comprising a first bicyclic structure consisting of a phenyl ring fused to a 6-membered heterocycle (oxygenated), substituted with a phenyl ring. The flavonoid content is expressed as % by weight relative to the total weight of the dry extract, and is measured by methods well known to persons skilled in the art, such as the chromatographic methods described notably by Mai (J. Sci. 2010; 37(3): 489-497, see more specifically section 2.5, page 491) and by Singh et al. (International Journal of Pharmacy and Pharmaceutical Sciences, Vol. 7, Issue 2, 2015, pp 144-147, see more specifically the section “Total flavonoid content”, page 145).

Preferably, the “polyphenol-rich extract of Vitex negundo” or the “polyphenol-enriched extract of Vitex negundo” is enriched in derivatives of dicaffeoylquinic acid (DCQ), notably 3,5-Di-Caffeoyl-Quinic acid (3,5-DCQ) and 4,5-Di-Caffeoyl-Quinic acid (4,5-DCQ).

The term “dicaffeoylquinic acid” (which may be abbreviated “DCQ” in the present description), refers to a diester composed of a quinic acid molecule in which two of the four alcohol functions have been esterified with caffeic acid. Dicaffeoylquinic acids are therefore acids having the general formula (I):

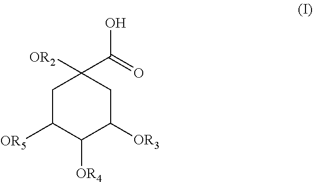

in which any two of radicals R₂, R₃, R₄ and R₅ represent a caffeyl group, the other two representing a hydrogen atom.

The term “caffeyl group” refers to a radical having general formula (II), derived from caffeic acid:

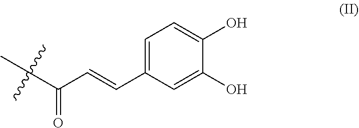

The various isomers of DCQ are therefore acids having general formula (I), with R₂, R₃, R₄ and R₅ as defined in the table below.

| Dicaffeoylquinic acids (DCQ) | R₂ | R₃ | R₄ | R₅ |
|---|---|---|---|---|
| 1,3-dicaffeoylquinic (1,3-DCQ) | Caffeyl | Caffeyl | H | H |
| 1,4-dicaffeoylquinic (1,4-DCQ) | Caffeyl | H | Caffeyl | H |
| 1,5-dicaffeoylquinic (1,5-DCQ) | Caffeyl | H | H | Caffeyl |
| 3,4-dicaffeoylquinic (3,4-DCQ) | H | Caffeyl | Caffeyl | H |
| also called isochlorogenic acid B | | | | |
| 3,5-dicaffeoylquinic (3,5-DCQ) | H | Caffeyl | H | Caffeyl |
| also called isochlorogenic acid A | | | | |
| 4,5-dicaffeoylquinic (4,5-DCQ) | H | H | Caffeyl | Caffeyl |
| also called isochlorogenic acid C | | | | |

In the present invention, the term “part” of a plant refers to any constituent part of a plant such as the roots, stem, leaves, fruit, skin, seeds or stone.

In the context of the invention, the expression “aeroponic cultivation of plants” refers to a soil-less cultivation method in which the plant roots are not in contact with a solid medium or even with a liquid medium: they are fed by a nutrient mist obtained by spraying (via an atomiser) the nutrient solution in a closed environment, as described for example in international application WO 01/33942.

The term “root exudation” refers to the recovery of the metabolites contained in the plant roots by means of a liquid brought into contact by percolation, spraying or immersion with the roots, still attached to the live or freshly cut plant (for less than 24 hours, and preferably as soon as possible after cutting, ideally just after cutting). In particular, root exudation can be carried by macerating the plant roots, freshly cut and/or still attached to the live plant, in an appropriate solvent and for an appropriate duration.

In the present invention, the expression “cosmetically acceptable” refers to that which is useful in the preparation of a cosmetic composition which is generally safe, non-toxic and neither biologically nor otherwise undesirable and which is acceptable for cosmetic use, notably in humans.

In the present invention, the expression “dermatologically acceptable” refers to that which is useful in the preparation of a dermatological composition which is generally safe, non-toxic and neither biologically nor otherwise undesirable and which is acceptable for dermatological use, notably in humans.

### DETAILED DESCRIPTION

Firstly, the present invention relates to a cosmetic or dermatological composition containing a polyphenol-rich extract of Vitex negundo as active principle, and, if need be, an appropriate excipient, which is notably dermatologically or cosmetically acceptable.

The polyphenol-rich extract of Vitex negundo of the compositions of the invention is preferably in liquid form. However, the proportions of the various constituents (notably the polyphenols, and more particularly the DCQs) of the extract are expressed relative to the weight of the “dry” extract. This “dry” extract is typically obtained by evaporation of the solvent in an oven at 105° C., until a constant-weight extract is obtained. The “dry” extract thus obtained is therefore essentially free of volatile substances, notably solvents and in particular water.

The polyphenol-enriched extract of Vitex negundo is advantageously obtained by a process using the Plant Milking® technology of the company Plant Advanced Technologies (technology described notably in international application WO 01/33942), which allows successive extractions from the plant's roots (milking) without destroying it. In this context, the plants are cultivated aeroponically and are fed by spraying the roots with a solution of essential, nutritive salts (nitrogen—N, phosphorous—P, potassium—K), precisely developed in terms of proportions and concentration, in order to obtain maximum root development, without altering the survival of the plant. The plant is occasionally stressed and/or elicited by appropriate substances, allowing a better production of molecules identified as being of interest, in this case polyphenols, and notably DCQs, in particular 4,5-DCQ.

The polyphenol-rich extracts of Vitex negundo of the compositions of the invention are obtained according to a process comprising the following successive steps:
a) aeroponic cultivation of Vitex negundo;
b) preferably, stimulation of the plant roots;
c) extraction by root exudation of the plants;
d) isomerisation of the dicaffeoylquinic acids obtained to 4,5-dicaffeoylquinic acid, and
e) purification and concentration of the extract thus obtained by successive filtrations, in particular by nanofiltration and/or sterilising filtration.

The skilled person, using his general knowledge, knows how to adapt the proportions and concentrations of various mineral salts to optimise root development and the concentration of molecules of interest. The mineral salt concentrations of the nutrient solutions are advantageously comprised in an electrical conductivity range from 0.2 to 1.6 mS.

Step a) is advantageously performed for a period ranging between 1 week and 8 weeks.

Step b) of stimulation makes it possible to significantly increase the metabolite content in the roots and thus to promote the flow of metabolites from the roots to the solvent chosen for maceration, without total loss of plant viability so that it can be reused. In other words, the plant stimulation step promotes the content and the secretion of molecules of interest.

Advantageously, step b) of stimulation is performed by spraying the plant with a solution of elicitors selected from jasmonic acid derivatives, ethylene generators, chitins, chitosans and mixtures thereof. In addition, step b) is advantageously performed for a period ranging between 1 day and 2 weeks.

Step b) can be performed simultaneously with or after step a). If step b) is performed simultaneously with step a) (in this case, preferably at the end of step a), which is longer), the stimulation solution can be added to the nutrient solution.

The plants thus cultivated are then subjected to a step c) of extraction by root exudation under conditions defined in terms of extraction solvent, pH, and extraction time, in order to obtain an extract rich in molecules of interest, i.e., polyphenols, notably dicaffeoylquinic acids. This step c) allows the recovery of the metabolites (DCQs) released by the plant roots by means of a liquid brought into contact by percolation or immersion with the roots.

Advantageously, step c) of root exudation is performed by maceration of the roots for a period ranging between 30 minutes and 96 hours, notably between 12 hours and 72 hours, more particularly between 24 hours and 48 hours. The duration of root exudation is selected so as to promote extraction of a high content of compounds of interest while not altering the survival of the plant and without leading to depletion of resources. Extraction is advantageously performed at acidic pH.

Step d) of isomerisation is performed according to techniques known to persons skilled in the art, in particular by successive readjustment of the pH of the extract (extraction at acidic pH, readjustment to basic pH by a strong basic solution and final readjustment to acidic pH by the addition of a strong acid). Step d) makes it possible to obtain a Vitex negundo extract characterised in that the majority of the dicaffeoylquinic acids that it contains are in the form of the 4,5-O-dicaffeoylquinic isomer.

Lastly, the extract obtained following step d) is subjected to a final step e) of purification and concentration by successive filtrations, in particular by nanofiltration and/or sterilising filtration. This step makes it possible to obtain the final polyphenol-rich, particularly DCQ-rich, extract of Vitex negundo.

The extract thus obtained may be in liquid or solid form. The extract in solid form is advantageously obtained by drying the liquid extract, according to the methods known to persons skilled in the art, such as atomisation or lyophilisation, for example, with or without carrier such as maltodextrin.

The polyphenol-rich extract of Vitex negundo of the compositions of the invention advantageously contain at least 5%, for example between 5% and 10%, in particular at least 6%, more particularly at least 7%, by weight of polyphenols, expressed as gallic acid equivalent, relative to the total weight of the dry extract.

In other words, the polyphenol-rich extract of Vitex negundo of the present invention advantageously contains at least 7% by weight of polyphenols, the percentages being expressed relative to the total weight of the dry extract of said extract (before optional addition of a drying carrier), or 1.3 mg of polyphenols per mL of liquid extract, and more specifically at least 5% by weight of total DCQs, or 1.1 mg of DCQ per mL of liquid extract.

Among the polyphenols present in this extract, the DCQs are of particular interest. Therefore, in an advantageous variant of the invention, the extract according to the invention can be characterised by its content of DCQ, and more particularly a mixture of the various isomers thereof, mainly comprising 4, 5-DCQ.

The polyphenol-enriched extract of Vitex negundo thus advantageously contains at least 1.5%, in particular at least 2.5%, more particularly at least 4%, in particular at least 5%, preferably at least 6%, by weight of dicaffeoylquinic acids, relative to the total weight of the dry extract. Said dicaffeoylquinic acids are in particular the isomers of 3,4-dicaffeoylquinic acid (3,4-DCQ), 3,5-dicaffeoylquinic acid (3,5-DCQ) and 4,5-dicaffeoylquinic acid (4,5-DCQ). Preferably, in the polyphenol-enriched extract of Vitex negundo, at least 40% by weight of the dicaffeoylquinic acids are in the form of the 4,5-dicaffeoylquinic isomer, relative to the total weight of dicaffeoylquinic acids.

The polyphenol-enriched extract of Vitex negundo obtained by the above process has the feature of containing a very small amount of flavonoids, usually present in large amounts in the Vitex negundo extracts of the prior art. Notably, the Vitex negundo plant cultivated under the abovementioned conditions synthesises and secretes a very small amount of flavonoids such as vitexin and derivatives thereof.

Therefore, the polyphenol-enriched extract of Vitex negundo contains less than 0.5% by weight, preferably less than 0.2%, in particular less than 0.1%, more particularly less than 0.05%, notably less than 0.01%, by weight of flavonoids, relative to the total weight of the dry extract.

According to an advantageous variant of the invention, the cosmetic or dermatological composition contains 0.001 to 10%, typically 0.01 to 5%, by weight of polyphenol-rich extract of Vitex negundo relative to the total weight of the composition. In these compositions, the polyphenol-rich extract of Vitex negundo is preferably in liquid form.

Depending on the type of administration desired, the composition and/or the active compounds of the invention can further contain at least one dermatologically acceptable excipient, or one cosmetically acceptable excipient. According to the first variant, an excipient suitable for administration via the external topical route, notably on the skin, skin appendages and/or mucous membranes, in particular skin, skin appendages and/or mucous membranes that are sensitive or damaged by the environment, for example by UV rays or pollution, is used.

The composition will be advantageously in the form of a preparation suitable for topical administration, notably including creams, emulsions, milks, ointments, lotions, oils, aqueous or hydro-alcoholic or glycolic solutions, powders, patches, sprays, shampoos, varnishes or any other product for external application.

The composition of the present invention can further contain at least one dermatological or cosmetic adjuvant known to persons skilled in the art, selected for example from thickeners, preservatives, fragrances, dyes, chemical or mineral filters, moisturising agents, thermal waters, etc.

The composition can further contain at least one other active compound in addition to the polyphenol-rich extract of Vitex negundo.

The invention also relates to a composition of the invention for use in preventing and/or treating:
disorders or diseases of the skin and/or mucous membranes and/or skin appendages,
vascular disorders, and/or
age-related disorders.

In particular, the composition of the invention is intended for the prevention and/or treatment of inflammatory reactions, oxidation reactions, disorders related to radical attacks linked or not to pollution, disorders of the barrier or homeostasis of the skin, skin appendages (hair and nails) and/or mucous membranes (gums, periodontal structures, genital mucosa) whether immature, normal or mature/old.

In particular, the compositions of the invention are useful as anti-ageing treatment, notably against chronological and actinic ageing of the skin, mucous membranes or skin appendages.

Advantageously, the composition of the invention can be used for the prevention and/or treatment of reactions, disorders or diseases:
of the skin, notably rosacea or facial erythrosis, sensitive skin, reactive skin, dry skin (xerosis), dehydrated skin, skin with redness, cutaneous erythema, old or photoaged skin, photosensitised skin, pigmented skin (melasma, post-inflammatory pigmentation, etc.), loose skin;
of the mucous membranes, such as gums and periodontal structures with possible gingivitis (sensitive gums of newborns, hygiene problems due to smoking or other), periodontal disease, or genital mucosa with possible irritation of the male or female external or internal genitalia, and/or
disorders related to inflammatory and radical reactions caused by UV exposure, and/or to pollutants or intrinsic reactions thus causing accelerated ageing, barrier disorders, vascular disorders, redness, etc.

The composition of the invention is also particularly useful in the prevention and/or treatment of vascular disorders, in particular to protect blood vessels and/or to act on blood circulation, notably blood microcirculation. The vascular disorder is typically selected from: facial erythrosis, cutaneous erythema, rosacea, pruritis, reactive skin and/or mucosa, with redness, in particular due to dilation of the subcutaneous capillaries. Notably, the composition of the invention is useful in the prevention and/or treatment of redness and facial erythrosis, and in the prevention and/or treatment of skin ageing related to intrinsic or extrinsic causes.

The composition of the invention is thus advantageously used in the prevention and/or treatment of dilation (chronic) of subcutaneous capillaries which can occur in conditions such as facial erythrosis, cutaneous erythema, rosacea, pruritus, reactive skin and/or mucosa, with redness, in particular due to dilation of the subcutaneous capillaries.

The composition of the invention is also advantageously used as a product against chronological or photoinduced ageing, in particular in the prevention of ageing, and of photo-induced ageing.

The composition of the invention is also advantageously used as a wound healing product, in the prevention and/or treatment of disorders related to wound healing and cutaneous organisation.

Disorders related to wound healing and cutaneous organisation refer to disorders resulting from the processes of wound healing and cutaneous organisation of the skin such as loose skin, stretch marks, dry patches, chapping, cracks in particular of the breasts.

In particular, the composition of the invention is used as a moisturising product in the prevention and/or treatment of disorders of the barrier or homeostasis of the skin, skin appendages (hair and nails) and/or mucous membranes (gums, periodontal structures, genital mucosa) whether immature, normal or mature/old.

Disorders of the barrier of the skin, skin appendages and/or mucous membranes refer to disorders of the outer layer of the epidermis.

Disorders of the homeostasis of the skin, skin appendages and/or mucous membranes refer to disorders resulting from processes of cell renewal and equilibrium such as psoriasis, diaper rash, atopic dermatitis, dry skin (xerosis), dehydrated skin and photosensitised skin.

The composition of the invention is also advantageously used in the treatment and/or prevention of inflammations due to rays of all kinds, in particular sunburn.

The composition of the invention is also advantageously used as a depigmenting or lightening product, notably in the treatment of pigmented skin (melasma, post-inflammatory pigmentation, etc.), or to reduce age spots for example.

The invention also relates to a cosmetic care method for the skin and/or skin appendages and/or mucous membranes, for improving the condition and/or appearance thereof, comprising or consisting in the administration, preferably via the topical route, of a composition of the present invention.

In particular, the invention relates to a cosmetic care method for the skin, for preventing the ageing thereof, comprising or consisting in the application to the skin of a composition of the present invention.

Advantageously, in the cosmetic care method of the invention, the composition of the invention is used as a product against chronological or photoinduced ageing, or as a moisturising product. In particular, in the cosmetic care method of the invention, the composition of the invention is used as a depigmenting or lightening product, notably in the treatment of pigmented skin (melasma, post-inflammatory pigmentation, etc.), or to reduce age spots for example.

The optimal modes of administration, dosing regimens and pharmaceutical forms of the compounds and compositions of the invention can be determined according to the criteria generally taken into account in the establishment of a dermatological and/or cosmetic treatment adapted to a patient, such as for example the patient's age or weight, the seriousness of his general condition, the tolerance to the treatment, the side effects observed, the skin type.

The following examples are given by way of illustration and in no way limit the scope of the present invention.

### EXAMPLES

I. Preparation of a Polyphenol-Enriched Extract of Vitex negundo of the Invention

A polyphenolic extract of Vitex negundo enriched in polyphenols, notably in DCQ, is obtained according to the following process:
a) Aeroponic cultivation of Vitex negundo according to the process patented by PAT with a defined nutrient solution (having the composition 15/10/30 N/P/K with an electrical conductivity ranging between 0.2 and 1.6 mS);
b) Stimulation of the plant for a period of one week with an elicitor as described above;
c) Extraction of the molecules of interest by maceration of the plant roots in a 70/30 propylene glycol/water mixture, for a period of two times 48 hours at room temperature and a pH of 1.6; at the end of extraction, the propylene glycol concentration is readjusted to 60%;
d) The solution obtained undergoes successive pH adjustment steps to arrive at a final pH of 3.5;
e) The extract is purified (removal of salts by nanofiltration) and concentrated by successive filtrations including nanofiltration and sterilising filtration.

The liquid polyphenol-enriched extract of Vitex negundo thus obtained has the following features (% of dry extract (DE), obtained by evaporation of the solvent in a ventilated oven at 105° C. until a constant-weight extract is obtained):
Dry extract: 1.8% (m/m)
Total polyphenol content (gallic acid equivalent): 9.5%/DE
Total Di-Caffeoyl Quinic derivative content (HPLC): 6.5%/DE
4, 5-DCQ content (HPLC): 3.3%/DE
Ash content: 14.5%.

Comparison with a “Conventional” Vitex negundo Extract

An extract of dry roots and dry leaves derived from a young Vitex negundo plant cultivated in soil according to conventional techniques (called “nonPAT extract”), but in the same environment (greenhouse), is obtained according to the following methods:
a) The dry roots or leaves of Vitex negundo are ground
b) The raw materials obtained are extracted separately at room temperature in a 70/30 glycerol/water mixture at pH 1.6,
c) The extract is purified by filtrations.

This extract is compared with a Vitex negundo extract (called “PAT extract”) obtained according to the above process comprising steps a, b, c and e, but not comprising step (d) of isomerisation.

The assay values obtained by HPLC are as follows (%, concentration of molecules of interest/dry extract of the total extract obtained):

| | | Conventional cultivation | |
|---|---|---|---|
| | PAT extract | (Non-PAT extract) | |
| | Roots | Dry roots | Dry leaves |
| Flavonoids (HPLC) | 0 | 1.7 | 1.1 |
| Hydroxycinnamic | 8.6 | 5.0 | 2.2 |
| derivatives (Total DCQs) | | | |

It can be seen that the PAT extract does not contain flavonoids. But specifically, it contains a much higher concentration of polyphenols, notably of hydroxycinnamic derivatives.

II. Biological Activities

In the following tests (except for the skin explant tests in section 11.4), a liquid extract of Vitex negundo of the invention, obtained according to the process of Example 1 (process comprising step d) of isomerisation), is used. It is subsequently tested at various dilutions. The Vitex negundo extracts of the invention are then characterised by their concentration relative to the concentration of the initial extract, the concentration being expressed as % of the corresponding dry extract introduced.

II.1. Preliminary Screening of Activity on Dermal Fibroblasts and Melanised Reconstructed Epidermises

The potential biological activities of the polyphenol-enriched extract of Vitex negundo were investigated using a gene expression modulation test on dermal fibroblasts and melanised reconstructed epidermises. Thus, the expression of 95 genes of major interest in skin and cosmetic physiology was studied by PCRarray on fibroblasts and melanised reconstructed epidermises.

a. Materials and Methods:

The polyphenol-enriched extract of Vitex negundo (0.01%, dry matter) was added to the culture medium of the fibroblasts or the reconstituted melanised human epidermises for 6 hours and 24 hours.

Expression of the selected markers was evaluated by quantitative RTPCR (microfluidic card). The variation in expression of the markers studied relative to the control was expressed as relative expression (RQ, RQ>1: increase, RQ<1: decrease).

b. Results

The most significant results are presented in the table below and suggest that the polyphenol-enriched extract of Vitex negundo, by varying the gene expression of certain markers, appears to be of particular interest in the following activities:
Healing and Strengthening of the Dermal Matrix:

Urokinasetype plasminogen activator (PLAU): plays a role in keratinocyte migration during the re-epithelialisation process in skin healing;

Dermatopontin (DPT): constituent of the dermal matrix, plays a role in fibroblast adhesion and activates collagen fibrillogenesis;

CYR61 protein: plays a role in dermal matrix remodelling by reducing the production of dermal matrix component, by activating metalloproteinases and by activating the expression of pro-inflammatory cytokines.

Antimicrobial, Antioxidant and Anti-inflammatory Defences:

Haem oxygenase 1 (HMOX1): involved in the degradation of haem, protective role against oxidative stress;

Lactotransferrin (LTF): has antimicrobial activity due to its ability to bind iron, stimulates re-epithelialisation, keratinocyte and fibroblast proliferation, and activates the synthesis of extracellular matrix components;

Prostaglandin G/H synthase 2 (PTGS2): enzyme which catalyses prostaglandin synthesis, marker involved in activation of inflammation.

The Dermoepidermal Junction:

Collagen 7 alpha 1 (COL7A1): component of the dermo-epidermal junction, forms fibrils allowing the basal lamina to attach to the dermis.

Laminin beta-3 subunit (LAMB3): constituent of the basal lamina which allows the basal lamina to attach to the epidermis.

The exfoliation Process:

Kallikrein 5 (KLKS): involved in maturation of the corneal layer.

Variations in the Expression of Genes of Interest in Fibroblasts and Reconstructed Epidermises.

| | | Reconstructed |
|---|---|---|
| 0.01% polyphenol-enriched | Dermal fibroblasts | epidermises |
| extract of Vitex negundo | Relative expression (RQ) | |
| Urokinase-type plasminogen | | 2.00 |
| activator (PLAU) | | (Increase >+100%) |
| CYR61 protein | 0.61 | |
| | (Decrease >+20%) | |
| Dermatopontin | 1.47 | |
| | (Increase >+20%) | |
| Haem oxygenase | 1.63 | |
| (decycling) 1 | (Increase >+20%) | |
| Lactotransferrin | | 1.68 |
| | | (Increase >+20%) |
| Prostaglandin G/H synthase 2 | 0.22 | |
| | (Decrease >+50%) | |
| Kallikrein 5 | | 1.40 |
| | | (Increase >+20%) |
| Collagen 7 alpha 1 | 1.26 | |
| | (Increase >+20%) | |
| Laminin beta-3 subunit | | 1.74 |
| | | (Increase >+20%) |

II.2. Dermal Matrix

a. Introduction

In order to study the potential of the polyphenol-enriched extract of Vitex negundo on the dermal matrix and the ageing thereof, its effect on the level of gene expression of extracellular matrix (ECM) markers was evaluated. The study was performed on normal dermal fibroblasts. The effect of the polyphenol-enriched extract of Vitex negundo was also evaluated on fibroblasts as a function of age using cells from a 28-year-old donor, from a 54-year-old donor and on fibroblasts from the 28-year-old donor, made senescent in vitro by successive replications.

b. Materials and Methods

Normal human fibroblasts were treated for 24 hours and 48 hours with 0.005% and 0.01% polyphenol-enriched extract of Vitex negundo. In parallel, a control (control cells) was prepared by treating fibroblasts with monopropylene glycol (extraction solvent). The gene expression of the selected markers was analysed by quantitative realtime RTPCR.

The results were statistically analysed by Student's t-test:

ns p>0.05: not significant, *0.01<p<0.05: significant, **0.001<p<0.01: very significant, ***p<0.001: highly significant.

The level of gene expression was expressed as relative quantity (RQ) and the effect of the treatment relative to the control cells as percentage increase.

c. Results

c.1. “Young” Fibroblasts

The study was performed on normal human dermal fibroblasts from a 28-year-old donor used in sections II.4 and II.5. The results presented below correspond to a treatment duration of 48 hours.

Gene Expression of Dermal Matrix Markers by “Young” Fibroblasts

### Results Expressed as Relative Quantity

| | | 0.005% Vitex | 0.01% Vitex negundo |
|---|---|---|---|
| | Control cells | negundo extract | extract |
| Elastin | 1.00 | 1.72 (+72% *) | |
| Fibrillin | 1.00 | | 1.57 (+57% *) |
| Decorin | 1.00 | | 1.55 (+55% *) |
| Collagen VII | 1.00 | | 1.51 (+51% *) |

The polyphenol-enriched extract of Vitex negundo significantly increased the gene expression of elastin, fibrillin, decorin and collagen VII. All these proteins are structural proteins of the dermal matrix. Elastin and fibrillin are markers involved in the elasticity of the dermis. Decorin controls the proper assembly of the collagen network. Collagen VII is a marker present in the dermo-epidermal junction and allows the matrix to be firmly anchored to the epidermis. By increasing these markers, the polyphenol-enriched extract of Vitex negundo thus seems to help maintain a proper structure of the dermis.

c.2. “Old” Fibroblasts

The study was performed on normal human dermal fibroblasts from a 54-year-old donor used in sections II.4 and II.5. The results presented below correspond to a treatment duration of 24 hours.

Gene Expression of Dermal Matrix Markers by “Old” Fibroblasts

### Results Expressed as Relative Quantity

| | Control | 0.005% Vitex | | 0.01% Vitex | |
|---|---|---|---|---|---|
| | cells | negundo extract | | negundo extract | |
| Collagen III | 1.00 | | | 1.37 | (+37%**) |
| Collagen IV | 1.00 | | | 1.30 | (+30%***) |
| Decorin | 1.00 | 1.51 | (+51% ns) | 1.44 | (+44% ns) |
| Dermatopontin | 1.00 | 1.35 | (+35%*) | 1.27 | (+27%**) |
| Metalloproteinase 1 | 1.00 | 0.49 | (−51%*) | 0.66 | (−34%**) |
| (MMP1) | | | | | |

The polyphenol-enriched extract of Vitex negundo significantly increased the gene expression of collagen III, decorin, dermatopontin, collagen IV and significantly decreased marker MMP1. All these proteins participate in the development of the dermal matrix. As described above, decorin controls the proper assembly of the collagen network and collagen IV, like collagen VII, is a marker present in the dermoepidermal junction which allows the proper anchoring of the matrix the epidermis. MMP1, for its part, is an enzyme which catalyses the degradation of collagens. By inhibiting MMP1 and increasing other markers, the polyphenol-enriched extract of Vitex negundo thus seems to promote the synthesis and the proper structure of the dermal matrix.

c.3. “Senescent” Fibroblasts

The study was performed on normal human dermal fibroblasts from a 28-year-old donor. The fibroblasts were aged in vitro by successive replications. Senescence was verified by evaluation of senescence markers (increase in the marker betagalactosidase, increase in the gene marker p21(+145%)). The results presented below correspond to a treatment duration of 24 hours.

### Gene Expression of Dermal Matrix Markers by “Senescent” Fibroblasts Results Expressed as Relative Quantity

| | | 0.005% | 0.01% Vitex |
|---|---|---|---|
| | Control cells | Vitex negundo extract | negundo extract |
| Decorin | 1.00 | 1.24 (+24%*) | 1.26 (+26%*) |
| Collagen VII | 1.00 | | 1.37 (+37% ns) |
| Sirtuin | 1.00 | | 1.18 (+18%*) |

The polyphenol-enriched extract of Vitex negundo increased the gene expression of decorin, collagen VII and sirtuin 1. The first two markers are involved in the structure of the dermal matrix and its proper attachment to the basal lamina. Sirtuin 1 is a longevity marker that delays cell senescence. By increasing these markers, the polyphenol-enriched extract of Vitex negundo thus seems to help maintain a proper structure of the dermal matrix and to slow cell senescence.

d. Conclusions

A positive effect of the polyphenol-enriched extract of Vitex negundo on the dermal matrix was shown on “young”, “old” and “senescent” fibroblasts. The Vitex negundo extract thus seems to help maintain a proper structure of the dermal matrix and thus to help protect the skin from skin ageing.

II.3. Antioxidant and Anti-inflammatory Action

a. Evaluation of the Antioxidant Effect of the Vitex negundo Extract

The antioxidant effect of the polyphenol-enriched extract of Vitex negundo is evaluated using the incorporation of DCFHDA (2′,7′-dichlorofluorescin diacetate) into keratinocytes in culture. This molecule is a nonfluorescent marker in an unoxidised state. Under oxidising conditions (in this case stress induced by hydrogen peroxide, H2O2), DCFHDA will be degraded to DCF, a molecule which will emit fluorescence. The fluorescence thus measured will be proportional to the amount of reactive oxygen species (ROS) produced by the cell in the presence of H₂O₂ and/or active agent.

a.1. Materials and Methods

Normal human keratinocytes were preincubated for 24 hours in the presence of 0.0005%, 0.001% and 0.005% (w/v) concentrations of the polyphenol-enriched extract of Vitex negundo, and 0.016%, 0.03% and 0.16% monopropylene glycol (controls corresponding to the solvent concentrations contained in the concentrations of Vitex negundo extract studied), 10 μM quercetin or 500 μM vitamin C (the latter two molecules serving as antioxidant reference).

The cells are then treated for 1 hour in the presence of 0.5 mM DCFH-DA.

Oxidation is induced by addition of 100 μM H₂O₂ for 20 minutes. A second treatment with the tested products is performed simultaneously with H₂O₂ stress (at the same concentrations as the pretreatment).

Lastly, a measurement of fluorescence density (DFU) corresponding to the amount of ROS is performed using a microplate reader.

In parallel, the number of biologically active cells was determined by a neutral red assay. The amount of ROS (reactive oxygen species) measured for each condition was normalised to the number of living cells determined by the neutral red assay.

The significance of the results was verified by a one-factor analysis of variance followed by Tukey's test (GraphPad PRISM software version 5.02, GraphPad Software, San Diego Calif. USA).

a.2. Results

### Production of Reactive Oxygen Species (ROS) in Keratinocytes Treated with Hydrogen Peroxide (H₂O₂)

***p<0.001; ns=not significant—one-factor ANOVA followed by Tukey's test

| | ROS (fluorescence units) | | | |
|---|---|---|---|---|
| | | Standard | | |
| | Mean | deviation | Increase (%) | Tukey |
| Control cells | 18850 | 2978 | | |
| 100 μM H₂O₂ | 139299 | 20667 | 639 | *** |
| 10 μmol/L quercetin | 24411 | 785 | −82 | *** |
| 500 μM vitamin C | 11940 | 872 | −91 | *** |
| 0.0005% Vitex negundo | 68328 | 8791 | −51 | *** |
| 0.001% Vitex negundo | 57668 | 6048 | −59 | *** |
| 0.005% Vitex negundo | 37036 | 2217 | −73 | *** |
| 0.016% MP glycol | 142134 | 12100 | 2 | ns |
| 0.03% MP glycol | 142005 | 15991 | 2 | ns |
| 0.16% MP glycol | 149469 | 14798 | 7 | ns |

An increase in ROS production was observed after H₂O₂ treatment, thus validating the model. Quercetin and vitamin C significantly decreased ROS production following H₂O₂ treatment. The antioxidant effect of these two references was indeed validated on the model.

The polyphenol-enriched extract of Vitex negundo at the three concentrations significantly decreased H₂O₂ stress-induced ROS production.

a.3. Conclusion

The polyphenol-enriched extract of Vitex negundo showed a strong antioxidant effect against H₂O₂induced stress.

b. Evaluation of the Anti-Inflammatory Effect of the Vitex negundo Extract

The inflammatory response is the body's normal, immediate and transient response to any environmental stress. However, under certain pathological or physiological conditions, this inflammatory reaction can be exacerbated and, if not controlled, can lead to tissue damage. In the skin, the keratinocyte is one of the first cells involved in the initiation of the inflammatory reaction in response to environmental stress.

The “stressed” keratinocyte will then release:
primary cytokines (IL1α, IL1β or TNFα) or secondary cytokines (IL8) that induce a cascade of reactions involving the immune system.
prostaglandins (PGE), which are members of the prostanoids family. The prostaglandin synthesis pathway that leads to the synthesis of PGE2 and other PGEs is induced by inflammatory stimuli.

The anti-inflammatory activity of the polyphenol-enriched extract of Vitex negundo was evaluated on a model of inflammation induced on keratinocytes by treatment with PMA (Phorbol 12-Myristate 13-Acetate). Release of the cytokines Interleukin-1 beta (IL1β) and Prostaglandin E2 (PGE2) was analysed.

b.1. Materials and Methods

Normal human keratinocytes were pretreated for 24 hours with 0.0005% and 0.001% concentrations of the polyphenol-enriched extract of Vitex negundo, 0.015% and 0.03% monopropylene glycol (solvent control), 0.1 μM dexamethasone and 0.1 μM indomethacin (the latter two references serving as antiinflammatory reference for the cytokines and prostaglandins, respectively).

Inflammation was then induced by addition of 10 μg/mL PMA overnight.

An assay of IL1β and PGE2 was then performed in the cell culture supernatants. In parallel, the number of biologically active cells was determined by a neutral red assay. The amount of cytokine measured for each condition was normalised to the number of living cells determined by the neutral red assay.

The significance of the results was verified by a one-factor analysis of variance followed by Tukey's test (GraphPad PRISM software version 5.02, GraphPad Software, San Diego Calif. USA).

b.2. Results

Assay of IL1β in Keratinocytes Treated with 10 μg/mL PMA

*p<0.05; **p<0.01; ***p<0.001 and ns=not significant—one-factor ANOVA followed by Tukey's test

| | IL1Beta (pg/mL)/OD neutral red | | | |
|---|---|---|---|---|
| | | Standard | | |
| | Mean | deviation | Increase (%) | Tukey |
| Control | 0.038 | 0.008 | | |
| PMA | 0.181 | 0.058 | 375 | *** |
| Dexamethasone + PMA | 0.120 | 0.014 | −34 | ns |
| Indometacin + PMA | 0.172 | 0.019 | −5 | ns |
| 0.0005% Vitex negundo | 0.092 | 0.005 | −49 | ** |
| 0.001% Vitex negundo | 0.088 | 0.018 | −51 | ** |
| 0.015% MP glycol | 0.140 | 0.014 | −23 | ns |
| 0.03% MP glycol | 0.128 | 0.016 | −29 | ns |

10 μg/mL PMA significantly increased the release of IL1β in the keratinocyte supernatants; therefore PMA indeed induced inflammation. 0.1 μM dexamethasone, as pretreatment for 24 hours, decreased the release of PGE2. The antiinflammatory effect of this reference was thus indeed validated.

The polyphenol-enriched extract of Vitex negundo, as pretreatment for 24 hours at the two concentrations, significantly decreased the release of 11143 and thus showed an anti-inflammatory action against PMA.

Assay of PGE2 in Keratinocytes Treated with 10 μg/mL PMA

*p<0.05; **p<0.01; ***p<0.001 and ns=not significant—one-factor ANOVA followed by Tukev's test

| | PGE2 (pg/mL)/OD neutral red | | | |
|---|---|---|---|---|
| | | Standard | | |
| | Mean | deviation | Increase (%) | Tukey |
| Control | 300.566 | 31.908 | | |
| PMA | 2360.965 | 155.001 | 686 | *** |
| Dexamethasone + PMA | 1339.332 | 183.082 | −43 | *** |
| Indometacin + PMA | 416.328 | 109.852 | −82 | *** |
| 0.0005% Vitex negundo | 1451.186 | 150.275 | −39 | ** |
| 0.001% Vitex negundo | 1679.102 | 92.887 | −29 | * |
| 0.015% MP glycol | 2017.886 | 163.757 | −15 | ns |
| 0.03% MP glycol | 2188.794 | 259.409 | −7 | ns |

10 μg/mL PMA significantly increased the release of PGE2 in the keratinocyte supernatants; therefore PMA indeed induced inflammation. 0.1 μM indometacin and dexamethasone, as pretreatment for 24 hours, significantly decreased the release of PGE2. The anti-inflammatory effect of these two references was thus indeed validated.

The polyphenol-enriched extract of Vitex negundo, as pretreatment for 24 hours at the two concentrations, significantly decreased the release of PGE2 and thus showed an anti-inflammatory action against PMA.

b.3. Conclusion

The anti-inflammatory effect of the polyphenol-enriched extract of Vitex negundo was demonstrated thanks to its action on the release of interleukin-1 beta and prostaglandin E2 in inflammatory conditions.

II.4. Antiageing Effect on Skin Explant

During skin ageing, an alteration of the dermis is commonly described. Following the results of the study of gene expression of the dermal matrix in fibroblasts (increase in elastin, fibrillin, collagen VII and decorin, among others), the potential antiageing effect of the polyphenol-enriched extract of Vitex negundo was evaluated on a skin explant model.

a. Formulation of the Vitex negundo Extract

For this test, the Vitex negundo extract (liquid) was used in a cream composition containing 0.08% or 2% by weight of said extract relative to the total weight of the cream.

b. Materials and Methods

Skin explants from a 46-year-old donor were treated topically or not (NT: control explant) with creams containing 0.8% and 2% polyphenol-enriched extract of Vitex negundo. One application daily for 5 days was performed. The explants were then fixed and imbedded in paraffin for immunofluorescence analysis.

Fluorescence quantification was performed using the Image J software. On average, 10 images per explant were taken.

c. Results

Protein Expression of Dermal Matrix Markers and of the Marker Klotho in Explants Treated with Vitex negundo (*p<0.05; **p<0.01; ***p<0.001; ns=not significant)

| Fluorescence | | | |
|---|---|---|---|
| units | Elastin | Collagen VII | Klotho |
| Control | 109.2 ± 0.6 | 67.7 ± 0.4 | 139.6 ± 1.0 |
| 0.8% V. Negundo | 111.2 ± 0.8 (ns) | 70.3 ± 0.6 (*) | 140.1 ± 2.1 (ns) |
| 2% V. Negundo | 111.7 ± 0.8 (*) | 69.9 ± 0.5 (**) | 143.2 ± 1.4 (*) |

d. Conclusions

The formulation with 2% polyphenol-enriched extract of Vitex negundo significantly increased the expression of elastin, collagen VII and Klotho.

Elastin is an essential component of the dermal extracellular matrix. This protein contributes in large part to the elasticity of the dermis. Collagen VII, for its part, is a marker of the dermo-epidermal junction. The latter anchors the dermis to the epidermis and thus helps maintain the general structure of the skin.

The Klotho protein is a protein involved in the ageing process and is thought to be related to longevity. Its increase would promote an induction of longevity and thus would delay skin ageing.

By stimulating these various markers, the polyphenol-enriched extract of Vitex negundo thus showed an antiageing effect on the explant model.

II.5. Protection Against DNA Damage

Many intrinsic and extrinsic factors, such as reactive oxygen species (ROS) or ultraviolet (UV) rays, induce DNA damage that can lead to significant cell damage, thus accelerating skin ageing. Mechanisms exist which can repair this DNA damage. However, during ageing, a decrease in DNA repair mechanisms has been observed. The ability of the Vitex negundo extract to induce activation of these mechanisms was studied on normal human fibroblasts.

a. Materials and Methods

Normal human fibroblasts were treated for 48 hours with 0.001% and 0.01% (w/v) Vitex negundo extract. In parallel, an extraction solvent control was prepared by treating fibroblasts with monopropylene glycol (control cells). The study was performed on normal human fibroblasts. After 48 hours of treatment, the cells are trypsinised and frozen.

From these cell pellets, nuclear extracts are prepared and a protein assay is performed.

The extracts are then deposited on a chip functionalised by plasmids containing specific series of DNA lesions:
8oxoG (p8oxoG)
alkylated bases (pEtheno)
glycols (pGlycols)
abasic sites (pAbas)
photoproducts (pyrimidine dimers and 6-4 photoproducts) (pCPD-64)
cisplatin adducts (pCisP)
benzo[a]pyrene adducts (pBPDE)

DNA repair enzymes contained in the extracts excise the lesions (or the DNA fragment flanking the lesions) and incorporate a marker (dNTP-biotin) during DNA resynthesis. The incorporated biotin is revealed by streptavidin coupled to a fluorochrome, Cy-5. The fluorescent signal is quantified using a scanner. It is proportional to the excision/resynthesis capacities of each extract with respect to each lesion. This test is used to characterise the base excision repair (BER), nucleotide excision repair (NER), and interstrand crosslink repair (ICLR) systems.

b. Results

Evaluation of Repair Pathways in Fibroblasts Treated with Vitex negundo

| Fluorescence units | p8oxoG | pAbas | pBPDE | pEtheno |
|---|---|---|---|---|
| Control cells | 2.5 · 10⁶ ± | 3.3 · 10⁶ ± | 5.4 · 10⁵ ± | 1.5 · 10⁶ ± |
| | 2.2 · 10⁵ | 1.2 · 10⁵ | 2.2 · 10⁴ | 5.5 · 10⁴ |
| 0.001% V. Negundo | 2.9 · 10⁶ ± | 3.5 · 10⁶ ± | 6.3 · 10⁵ ± | 1.8 · 10⁶ ± |
| | 1.5 · 10⁵ | 1.7 · 10⁵ | 9.3 · 10⁴ | 1.5 · 10⁵ |
| 0.01% V. Negundo | 2.7 · 10⁶ ± | 3.5 · 10⁶ ± | 6.3 · 10⁵ ± | 1.6 · 10⁶ ± |
| | 1.3 · 10⁵ | 1.1 · 10⁵ | 3.7 · 10⁴ | 1.5 · 10⁵ |

c. Conclusion

The polyphenol-enriched extract of Vitex negundo promotes the DNA repair pathways of 8-oxo-2′-deoxyguanosine (p8oxoG), abasic sites (pAbas), benzo[a]pyrene adducts (pBPDE) and alkylated bases (pEtheno). It thus seems to help reduce premature skin ageing induced by unrepaired DNA damage.

II.6. Activation of Innervation in Old Keratinocyte/Neuron Cocultures

The epidermis consists of many free nerve endings coming from sensory neurons of the spinal ganglia located along the spinal cord. These neurons have axons that transmit information such as touch, heat, pain, for example. The innervation density varies during ageing. Thus, the innervation of a given region is lower for an elderly person than for a young adult. This phenomenon leads to a decrease in the individual's ability to perceive his environment. One of the properties of sensory neurons is their ability to reinnervate skin regions that have partially lost this density of free endings. Keratinocytes release growth factors allowing neurons to emit free endings to the outermost layers of the epidermis. The aim of this study was to evaluate the potential of the Vitex negundo extract on the tropism of sensory neurons and their ability to emit projections in the presence of old keratinocytes.

a. Materials and Methods

Sensory neurons are derived from hiPS cells obtained from human fibroblasts. hiPS cells were cocultured with keratinocytes from a 33-year-old donor and a 59-year-old donor. The co-cultures were treated with the Vitex negundo extract at various concentrations (0.003%, 0.001%, 0.0005%, 0.0001%, 0.00005%, 0.00001% (w/v)), with 0.1% monopropylene glycol (extraction solvent control) and with 50 ng/mL nerve growth factor (NGF) serving as positive reference for dendrite elongation.

After 6 days of co-culture, the cells were fixed with 2% paraformaldehyde solution and then labelled with an anti-β-tubulin antibody. Labelling was observed using a fluorescence microscope and 20 images were acquired per co-culture. The length of the sensory neuron projections was measured and normalised by the number of labelled cell bodies. All statistics were done using the t-test and the ANOVA test (*p<0.05; **p<0.01; ***p<0.001).

b. Results

Evaluation of Neurite Length of Sensory Neurons in Neuron/Keratinocyte Co-Cultures Treated with Polyphenol-Enriched Vitex negundo (*p<0.05; **p<0.01; ***p<0.001; ns=not significant), Comparison of the 33-year-old donor versus the 59-year-old donor

| | Total neurite length | Neurite length per cell |
|---|---|---|
| | (arbitrary units) | (arbitrary units) |
| Control cells (33 years) | 10851 ± 2351 | 167 ± 11 |
| Control cells (59 years) | 6865 ± 633 (**) | 143 ± 16 (*) |

Total neurite length and neurite length per cell are significantly decreased in cocultures with the 59-year-old keratinocytes in comparison with the 33-year-old keratinocytes.

Evaluation of Dendrite Length in Neuron/59-year-old Keratinocyte Co-Cultures following Treatment with the Polyphenol-Enriched extract of Vitex negundo

| | Total neurite length | Neurite length per cell |
|---|---|---|
| | (arbitrary units) | (arbitrary units) |
| Control cells (59 years) | 6865 ± 633 | 143 ± 16 |
| 50 ng/mL NGF | 10393 ± 1849 (**) | 212 ± 23 (*) |
| 0.0005% Vitex negundo | 10152 ± 1351 (**) | 195 ± 21 (*) |
| 0.001% Vitex negundo | 7434 ± 970 (ns) | 178 ± 21 (ns) |
| 0.003% Vitex negundo | 10436 ± 2548 (**) | 224 ± 40 (***) |

50 ng/mL NGF significantly increased total neurite length and neurite length per cell. This result validates the model.

The polyphenol-enriched extract of Vitex negundo significantly increased total neurite length and neurite length per cell in human sensory neuron/old keratinocyte cocultures.

c. Conclusion

The polyphenol-enriched extract of Vitex negundo thus seems to help prevent the decline of the sensory neuron network in the skin observed during skin ageing and thus seems to protect the skin from a loss of sensations appearing with age.

II.7. Depigmenting Effect

The depigmenting effect of the compositions of the invention containing a Vitex negundo extract was evaluated against placebo during a clinical test.

In these tests, the placebo is a cream containing no cosmetic active agents (only cosmetic excipients), and the composition of the invention is a cream of the same composition as the placebo, further comprising 1% by weight of Vitex negundo extract of the invention.

| Composition of the placebo cream | % INCI |
|---|---|
| WATER | 80.876 |
| ISONONYL ISONONANOATE | 10.000 |
| BUTYLENE GLYCOL | 3.000 |
| CETYL ALCOHOL | 1.760 |
| GLYCERYL STEARATE | 1.260 |
| CAPRYLOYL GLYCINE | 0.800 |
| CARBOMER | 0.600 |
| PEG-75 STEARATE | 0.490 |
| SODIUM HYDROXIDE | 0.324 |
| CAPRYLYL GLYCOL | 0.300 |
| CETETH-20 | 0.245 |
| STEARETH-20 | 0.245 |
| DISODIUM EDTA | 0.100 |
| | 100.000000 |

a. Protocol

The test is performed on a panel of 2 groups of 25 subjects: Caucasian women of phototypes I to III, healthy, aged 40 to 60 years. Inclusion was decided according to various clinical scales.

It should be noted that D0 is the starting point of the study (before application of the cream containing the composition of the invention or the placebo).

Group I applied the composition of the invention to the face and hands, while group II applied a placebo to the face and hands. Both groups I and II applied the composition of the invention or the placebo, respectively, twice daily for 56 days.

The effect of the composition of the invention was evaluated by colorimetric measurements on the forehead, cheek, and a spot on the hands. The parameters measured are luminance (L*) (expressed in arbitrary units, a.u.) and individual typology angle (ITA).

The measurements were made with a spectrocolorimeter (Minolta) with a 3 mm head.

The parameter L* of the L*a*b* colour space gives information on the luminance of the skin colour measured.

L*: corresponds to lightness or luminance;

a*: represents the hue and saturation of the colour on a green-red colour axis;

b*: represents the hue and saturation of the colour on a blue-yellow colour axis.

The higher the value L*, the more luminous and light the skin colour.

The ITA gives information on skin tone. A high angle translates to lighter skin.
Measurement area: skin colour is measured on the cheek, forehead or hand on an area delimited beforehand and marked using a marking mask.
Application of the cream to be tested: the cream containing the composition of the invention or the placebo is applied to the entire hand or face like a usual cream.

b. Results

The results of measurement of L* and of ITA on D0 and D=56 (ΔL and ΔITA) are compared to determine the efficacy of the compositions of the invention for each of the areas tested.

On the hand: The active agent increases luminance and lightens the skin to a significantly greater degree compared to the placebo on a spot:

ΔL*(composition): +6%

ΔL*(placebo)=+3%

The difference between the two measurements is significant in favour of the composition of the invention.

ΔITA(composition): +48%

ΔITA(placebo)=+23%

The difference between the two measurements is significant in favour of the composition of the invention.

On the forehead: The active agent increases luminance and lightens the skin to a significantly greater degree compared to the placebo on the forehead:

ΔL*(composition): +3%

ΔL*(placebo)=+0.7%

The difference between the two measurements is significant in favour of the composition of the invention.

ΔITA(composition): +11%

ΔITA(placebo)=+1.2%

The difference between the two measurements is significant in favour of the composition of the invention.

On the cheek: The active agent increases luminance and lightens the skin to a significantly greater degree compared to the placebo on the cheek:

ΔL*(composition): +3%

ΔL*(placebo)=+1.5%

The difference between the two measurements is significant in favour of the composition of the invention.

ΔITA(composition): +15%

ΔITA(placebo)=+7%

The difference between the two measurements is significant in favour of the composition of the invention.

c. Conclusion

The compositions of the invention have a significant depigmenting and lightening effect compared to a placebo both on a spot on the hand and on the forehead or cheeks.

III. Cosmetic and Dermatological Compositions for Topical Application

Several compositions for topical application are presented below. The PAT extract of Vitex negundo (VN) of Example 1 can be incorporated into various cosmetic products, such as cleansing waters, oil-in-water emulsions, water-in-oil emulsions, oils, milks, lotions, shampoos, foaming products and sprays, the compositions of which are presented below by way of examples.

Anti-Ageing Emulsion

| Raw material/Trade name or INCI name | % |
|---|---|
| LIQUID ISOPARAFFIN | From 5 to 20% |
| ISOCETYL STEARATE | From 5 to 20% |
| AL-MG HYDROXYSTEARATE | From 5 to 20% |
| ABIL WE 09 | From 1 to 5% |
| GLYCEROL | From 1 to 5% |
| PETROLEUM JELLY | From 1 to 5% |
| MICRONISED ZINC OXIDE | From 1 to 5% |
| BUTYLENE GLYCOL | From 1 to 5% |
| RETINOL | From 0 to 1% |
| VITAMIN C | From 0 to 5% |
| Polyphenol-enriched extract of Vitex negundo of | From 0.01 to 10% |
| section I | |
| ISONONYL ISONONANOATE | From 1 to 5% |
| BEESWAX | From 1 to 5% |
| SODIUM TARTRATE | From 1 to 5% |
| SODIUM CHLORIDE | From 0 to 5% |
| GLYCINE | From 1 to 5% |
| PRESERVATIVES | From 0 to 1% |
| CHOLESTEROL | From 0 to 1% |
| PHYTOSPHINGOSINE | From 0 to 1% |
| TARTARIC ACID | From 0 to 1% |
| PURIFIED WATER | QS 100% |

Fortifying Hair Lotion

| Raw material/Trade name or INCI name | % |
|---|---|
| PURIFIED WATER | QS 100% |
| METHYL PROPANEDIOL | From 5 to 20% |
| PRESERVATIVE | From 0 to 2% |
| pH ADJUSTER | From 0 to 1% |
| FRAGRANCE | From 0 to 1% |
| BIOTIN | From 0 to 5% |
| VITAMIN B9 | From 0 to 5% |
| Polyphenol-enriched extract of Vitex negundo of | From 0.01 to 10% |
| section I | |
| BETA-SITOSTEROL | From 0 to 1% |
| ETHYLHEXYL COCOATE | From 0 to 5% |
| PEG-40 CASTOR OIL | From 0 to 5% |

Photoprotective-Anti-Ageing Stick

| Raw material/Trade name or INCI name | % |
|---|---|
| CASTOR OIL | QS 100% |
| OLEIC ALCOHOL | From 10 to 20% |
| PALM KERNEL OIL | From 10 to 20% |
| POLYGLYCERIN-3-BEESWAX | From 10 to 20% |
| CANDELILLA WAX | From 10 to 20% |
| HECTORITE | From 10 to 20% |
| TITANIUM DIOXIDE | From 0 to 5% |
| Polyphenol-enriched extract of Vitex negundo of | From 0.01 to 10% |
| section I | |
| SHEA BUTTER | From 0 to 5% |
| VITAMIN E | From 0 to 1% |

1-12. (canceled)

13. A cosmetic or dermatological composition containing:
as active principle a Vitex negundo extract, said extract containing at least 5% by weight of polyphenols, expressed as gallic acid equivalent, relative to the total weight of the dry extract, and said extract containing less than 0.05% by weight of flavonoids and at least 1.5% by weight of dicaffeoylquinic acid relative to the total weight of the dry extract, and
if need be, an appropriate excipient, which is notably dermatologically or cosmetically acceptable.

14. The composition of claim 13, wherein the Vitex negundo extract contains at least 2.5% by weight of dicaffeoylquinic acids, relative to the total weight of the dry extract.

15. The composition of claim 14, wherein, in the Vitex negundo extract, at least 40% by weight of the dicaffeoylquinic acids are in the form of the 4,5-dicaffeoylquinic isomer, relative to the total weight of dicaffeoylquinic acids.

16. The composition of claim 13, wherein the Vitex negundo extract contains less than 0.01% by weight of flavonoids, relative to the total weight of the dry extract.

17. The composition of claim 13, containing 0.001 to 10% by weight, advantageously 0.01 to 5% by weight, of said Vitex negundo extract, preferably in liquid form, relative to the total weight of the composition.

18. The composition of claim 13, wherein it is in the form of a preparation suitable for topical administration, notably including creams, emulsions, milks, ointments, lotions, oils, aqueous or hydroalcoholic or glycolic solutions, powders, patches, sprays, shampoos, varnishes or any other product for external application.

19. A method for preventing and/or treating:
disorders or diseases of the skin and/or mucous membranes and/or skin appendages, sensitive skin, and/or
skin ageing of intrinsic or extrinsic origin,
comprising administering to a subject in need thereof an effective amount of the composition of claim 13.

20. A method for the prevention and/or treatment of vascular disorders comprising administering to a subject in need thereof an effective amount of the composition of claim 13.

21. The method of claim 20, wherein the vascular disorder is selected from: facial erythrosis, cutaneous erythema, rosacea, pruritus, reactive skin and/or mucosa, with redness, in particular due to dilation of the subcutaneous capillaries.

22. A cosmetic care method for the skin and/or skin appendages and/or mucous membranes, for improving the condition and/or appearance thereof, comprising the administration, preferably topical, of a cosmetic composition as defined according to claim 13.

23. The cosmetic care method of claim 22, for acting against chronological or photo-induced ageing.

24. The cosmetic care method of claim 22, for depigmenting or lightening, notably for the treatment of pigmented skin or for reducing age spots.
